(19) United States
(12) Patent Application Publication
(10) Pub. No.: US 2026/0053883 A1
(43) Pub. Date: Feb. 26, 2026

(54) OXYTOCIN COMPOSITIONS FOR TREATMENT OF VISUAL SNOW SYNDROME

(71) Applicant: Maskbegone LLC, Summit, NJ (US)

(72) Inventors: Lawrence C Newman, Katonah, NY (US); James Cramer, Summit, NJ (US)

(73) Assignee: Maskbegone LLC, Summit, NJ (US)

(21) Appl. No.: 19/299,582

(22) Filed: Aug. 14, 2025

Related U.S. Application Data
(60) Provisional application No. 63/686,317, filed on Aug. 23, 2024.

(51) Int. Cl. A61K 38/095; A61K 9/00; A61K 9/08; A61K 47/02; A61K 47/12

(52) U.S. Cl. CPC A61K 38/095; A61K 9/0043; A61K 9/08; A61K 47/02; A61K 47/12

(57) ABSTRACT

In an embodiment, a method of treating visual snow syndrome is disclosed. The method includes administering a therapeutically effective amount of oxytocin, which may be present with a thickener, in a suitable form to the upper nasal cavity of an individual. A pharmaceutical composition of oxytocin, which may include a thickener, for intranasal delivery for treating visual snow syndrome is also disclosed.

### CROSS REFERENCE TO RELATED APPLICATIONS

This application claims priority of U.S. Provisional Application No. 63/686,317, entitled “OXYTOCIN COMPOSITIONS FOR TREATMENT OF VISUAL SNOW SYNDROME,” filed on Aug. 23, 2024, the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Feb. 8, 2024, is entitled “058636.00303_ST26.xml”, and is 1,797 bytes in size.

### BACKGROUND

Visual snow syndrome (VSS) is a disorder that causes a perception of “snow” or static, like the picture on an old television. VSS is subjectively perceptual, which means that only the affected subject can perceive the snow. The perceived sensation may appear as colorful, black and white, transparent, or flashing snow, and it may be intermittent or continuous. The cause of VSS is unknown, but it may be related to excitability in the occipital lobes of the brain (i.e., where images are processed). Some have postulated that VSS is the visual equivalent of tinnitus. People with tinnitus perceive noise (e.g., ringing, hissing, whistling) while those suffering from visual snow “see” static and other visual distortions (e.g., afterimages, light sensitivity, impaired night vision) in the absence of an external stimulus.

It is estimated that about 2% to 3% of people are affected by VSS. Severe or prolonged VSS can affect quality of life for the patient as well as their families. People affected by VSS may also experience migraines and tinnitus. VSS may also be associated with anxiety, depression, sleep and cognitive difficulties, dizziness, and vertigo as a result of misdiagnosis, misattribution to a psychogenic cause, or a lack of treatment options. Additional information on VSS can be found at https://my.clevelandclinic.org/health/diseases/24444-visual-snow-syndrome, and Visual Snow: Visual Misperception, https://journals.lww.com/jneuro-ophthalmology/fulltext/2018/12000/visual_snow_visual_misperception.20.aspx, the disclosures of which are incorporated herein by reference in their entirety.

Current therapies for VSS include use of lamotrigine (an anti-seizure medication), transcranial magnetic stimulation, and blue light blockers. However, these treatments do not provide satisfactory relief and may even be associated with significant adverse events (e.g., when using lamotrigine). Therefore, there continues to be a need to develop more effective therapies.

### SUMMARY

This disclosure provides methods for treatment of VSS. In an embodiment, a method of treating VSS comprises administering intranasally to an individual who is afflicted with VSS 50 to 260 IU of oxytocin over a period of 24 hours. The oxytocin may be delivered in a composition which comprises a thickener present in an amount of from 5% to 12% by weight of the composition. The thickener may have a viscosity of from 4,000 to 12,000 cps. The oxytocin may be in a powdered or aerosolized form and may be delivered to the upper nasal cavity of the individual. The composition may further comprise a mucoadhesive polymer. The oxytocin may be delivered in 3 to 5 doses over the 24 hour period, each dose delivering 12 to 65 IU of oxytocin.

In another embodiment, a method of treating VSS comprises delivering oxytocin to the upper nasal cavity of an individual at an amount of 20 to 225 IU of oxytocin over a 24 hour period. The oxytocin may be delivered in a composition which further comprises a mucoadhesive polymer and a thickener present in an amount of from 5% to 12% by weight of the composition. The thickener may have a viscosity of from 4,000 to 12,000 cps. The oxytocin may be delivered in a powdered or aerosolized form in 3 to 5 doses over the 24 hour period.

This disclosure also provides compositions for treatment of VSS. In an embodiment, a pharmaceutical composition for treating VSS comprises 100 to 600 IU per ml of the composition and a thickener present in an amount of from 5% to 12% by weight of the composition. The thickener may have a viscosity of from 4,000 to 12,000 cps. The pharmaceutical composition of claim 12, wherein the composition is in a powdered or aerosolized form and is delivered to the upper nasal cavity of an individual. The composition may further comprise a mucoadhesive polymer and be delivered in 3 to 5 doses over a 24 hour period. The composition may further comprise magnesium chloride, citric acid or sodium citrate.

In another embodiment, a sprayable unit dose formulated for intranasal administration and comprises 15 to 65 IU oxytocin to treat VSS. The sprayable unit dose may further comprise a thickener present in an amount of from about 5% to 12% by weight. The thickener may have a viscosity of from about 4,000 to 12,000 cps. The sprayable unit dose may comprise a volume of from 0.05 to 0.15 ml.

The foregoing summary is illustrative only and is not intended to be in any way limiting. In addition to the illustrative aspects, embodiments, and features described above, further aspects, embodiments, and features will become apparent by reference to the following detailed description.

### DETAILED DESCRIPTION OF THE INVENTION

The present disclosure provides compositions and methods for treatment of visual snow syndrome (VSS). The compositions comprise therapeutically effective doses of oxytocin for intranasal administration. The methods comprise administering intranasally to an individual in need of treatment a composition comprising a therapeutically effective amount of oxytocin. A therapeutically effective amount may be delivered in one or more doses.

Oxytocin is a nine amino acid cyclic peptide hormone with two cysteine residues that form a disulfide bridge between positions 1 and 6. The amino acid sequence for human oxytocin is Cys-Tyr-Ile-Gln-Asn-Cys-Pro-Leu-Gly (SEQ ID NO:1). The oxytocin peptide for use in the present methods can be natural or synthetic, or may be produced by recombinant technologies. Oxytocin is also commercially available.

Peptide analogs and derivatives of oxytocin are known in the art and can be used in the present methods. Some example of oxytocin analogs include 4-threonine-1-hydroxy-deaminooxytocin, 4-serine, 8-isoleucine-oxytocin, 9-deamidooxytocin, 7-D-proline-oxytocin and its deamino analog, (2,4-diisoleucine)-oxytocin, deamino oxytocin analog, 1-deamino-1-monocarba-E12-Tyr(OMe)]-OT(dCOMOT), carbetocin, 4-threonine, 7-glycine-oxytocin (TG-OT), oxypressin, deamino-6-carba-oxytoxin (dC60), L-371,257 and the related series of compounds containing an ortho-trigluoro-ethoxyphenylacetyl core such as L-374,943. Another example of an oxytocin derivative that can be used in the present methods is merotocin, which is also known as carba-1-(4-FBzlGly7)dOT. Merotocin may be designated as compound FE 202767, and is also referred to as glycinamide, N-(4-mercapto-1-oxobutyl)-L-tyrosyl-L-isoleucyl-Lglutaminyl-L-asparaginyl-L-cysteinyl-N-[(4-fluorophenyl)methyl]glycyl-L-leucyl-, cyclic (1, Üí5)-thioether, or [Bua1,4-FBzlGly7]oxytocin. Additional information regarding merotocin can be found in U.S. Patent Application Publication 2022/0313774 to Ferring (the description of merotocin is incorporated herein by reference). Additional examples of oxytocin analogs can be found in U.S. Patent Application Publication 2014/0066373 to Cai and in U.S. Pat. No. 8,673,841 to Ferring (the descriptions of which analogs in both are incorporated herein by reference).

Oxytocin or its analogs may be chemically modified, for example, by amidation, the use of D amino acids in the peptide, incorporation of small non-peptidyl moieties, as well as the modification of the amino acids themselves (e.g., alkylation or esterification of side chain R-groups). Such analogs, derivatives and fragments should at least substantially retain the desired biological activity of the native oxytocin peptide of SEQ ID NO:1. For example, in embodiments, such analogs may retain at least 70%, at least 80%, at least 90%, or at least 95% of the activity of oxytocin of SEQ ID NO:1.

Clinical practice and clinical trials revealed several ways in which using analogs and derivatives of oxytocin, particularly merotocin, for the treatment of VSS may be more effective than using oxytocin. Some advantages of using analogs or derivatives of oxytocin such as merotocin in the present methods include the fact that oxytocin analogs and derivatives (a) can be modified to make them temperature stable, thus ensuring consistent potency of the medication even at room temperature and eliminating the need for constant refrigeration, (b) have a longer half-life life than oxytocin, thus reducing the need for multiple daily doses, (c) can be administered in other forms besides nasal sprays, such as a powdered or aerosolized formulation, or a solution suitable for parenteral administration, and (d) target only the oxytocin V1 receptor, thus eliminating the possibility of off target side-effects.

The term “treatment” as used herein refers to reduction in one or more symptoms or features associated with the presence of the particular condition being treated. Treatment does not necessarily mean complete cure or remission, nor does it preclude recurrence or relapses. For example, treatment in the present disclosure means reducing one or more symptoms associated with VSS. Treatment can be orientated symptomatically, for example, to suppress symptoms. It can be effected over a short period, over a medium term, or can be a long-term treatment, such as, for example, within the context of a maintenance therapy. Administrations may be intermittent, periodic, or continuous.

The term “therapeutically effective amount” as used herein in reference to a single agent is the amount sufficient to achieve, in a single or multiple doses, the intended purpose of treatment. The exact amount desired or required will vary depending on the particular compound or composition used, its mode of administration, patient specifics, and the like. Appropriate effective amounts can be determined by one of ordinary skill in the art informed by the present disclosure.

Where a range of values is provided in this disclosure, it should be understood each intervening value to the tenth of the value of the lower limit, and all intervening ranges between the upper and lower limit of that range are encompassed within the invention, unless clearly indicated otherwise.

A reference to singular form also includes its plural form, and vice versa.

The present compositions may be delivered intranasally. The term “intranasal delivery” as used herein means the composition or active agent (e.g., oxytocin) is delivered to the nose, nasal passageways or nasal cavity. The oxytocin or compositions comprising oxytocin may be in the form of spray, drops, powder, suspension, gel, inhalant, aerosolized, or any other suitable form.

Compositions comprising oxytocin can be prepared for pharmaceutical use by incorporation with a pharmaceutically acceptable carrier or diluent. The compositions can be formulated for intranasal delivery. The formulations may comprise excipients, vehicles, emulsifiers, stabilizers, preservatives, mucosal adhesives, antibacterial agents, buffers, and/or other additives. General pharmaceutical formulation components and methods of preparation can be found in Remington's Pharmaceutical Sciences, 2000, 20th ed.

In an embodiment, oxytocin formulations for the present methods may be in the form of powder (such as a dry powder), liquid based, or aerosolized. When used in a powder form, the powder may be substantially pure oxytocin (such as at least 50%, 60%, 70%, 80%, 90%, 95%, or 99%), or may contain other components such as salts, and the like. In an embodiment, the powder may be 94-99% pure oxytocin. When used in a liquid based or aerosolized form, the composition may contain salts and buffers, and may optionally contain a thickener. The compositions may also optionally contain excipients, carriers, and the like. Oxytocin may be present as incorporated in nanoparticles, including liposomes.

In an embodiment, the oxytocin formulations may be liquid based or aerosolized and comprise a magnesium salt, such as, magnesium chloride or magnesium citrate, and the like.

In an embodiment, oxytocin is present as a sprayable composition. The sprayable composition (which may also be delivered as drops or aerosolized) may comprise or consist essentially of oxytocin and salts. In an embodiment, the composition comprises, consists essentially of, or consists of oxytocin, magnesium salt, sodium citrate, and citric acid. In an embodiment, the composition further comprises a mucoadhesive polymer. The sprayable compositions may be delivered using a sprayer. The sprayer may include an operating mechanism (e.g., a spray head or a pump) in fluid communication with a reservoir. The spray device may be manually actuated or may be pressurized.

The amount of oxytocin may be 100 to 600 IU per ml of the composition. In an embodiment, the amount of oxytocin per ml can be 150 to 450 IU. In various embodiments, the oxytocin can be 150, 200, 250, 300, 350, 400, or 450 IU per ml.

The magnesium salt may be any pharmaceutically acceptable magnesium salt. Examples include magnesium chloride, magnesium citrate, magnesium acetate, and the like.

A “mucoadhesive agent” (also referred to herein as a “thickener”) generally refers to compounds that adhere to the mucosal surface. Mucoadhesive agents may be polymers such as anionic polymers (such as alginate, xanthan gum, carageenan), cationic polymers (such as, chitosan), non-ionic polymers (such as guar gum), amphoteric polymers, synthetic polymers, and the like. In an embodiment, the mucoadhesive polymer is Mucolox™. These agents generally increase the viscosity of the composition. In an embodiment, a mucoadhesive agent may have a viscosity of from 4,000 to 12,000 cps. In an embodiment, the mucoadhesive agent has a viscosity of from 5,000 to 10,000 cps. In various embodiments, the viscosity of the mucoadhesive agent may be 5,000, 6,000, 7,000, 8,000, 9,000, 10,000, 11,000 cps.

In an embodiment, the present composition is present in a sprayable unit dose for intranasal administration comprising or consisting essentially of from 15 to 65 IU of oxytocin per unit dose. In an embodiment, the present composition is present in a sprayable unit dose for intranasal administration comprising or consisting essentially of from 15 to 65 IU of oxytocin per unit dose and a thickener present in an amount of from about 5% to 12% by weight of the composition, wherein the thickener has a viscosity of from about 4,000 to 12,000 cps. In an embodiment, the unit dose may comprise 20, 25, 30, 35, 40, 45, 50, 55, or 60 IU of oxytocin. The unit dose may be from 0.05 to 0.15 mls. In embodiments, the unit dose may be 0.05, 0.06, 0.07, 0.08, 0.09, 0.1, 0.11, 0.12, 0.13, 0.14, or 0.15 mls. In an embodiment, the unit dose is about 0.1 ml.

In an embodiment, the disclosure provides a pharmaceutical composition for intranasal administration comprising or consisting essentially of: a) from 100 to 600 IU of oxytocin per ml, optionally, b) a thickener present in an amount of from 5% to 12% by weight of the composition, wherein the thickener has a viscosity of from about 4,000 to 12,000 cps; and optionally, c) magnesium chloride, citric acid and sodium citrate.

In an embodiment, this disclosure provides a pharmaceutical composition suitable for intranasal administration comprising oxytocin, and a thickener (viscosity regulating agent) present in an amount of from about 1% to about 15% by weight of the composition. The thickener, such as Mucolox™ may be present in an amount of from 5% to 12% (having a viscosity of 4,000 to 12,000 cps). The oxytocin may be present in an amount of 0.05 to 0.1% by weight. The oxytocin may be from 100 to 600 IU per ml. The composition may comprise salts, such as magnesium salts, zinc salts, sodium salts, and the like, and buffers. In an embodiment, the composition may comprise oxytocin present in an amount of 0.05% to 0.1% by weight; a thickener in an amount of about 5% to 12% by weight, said thickener having a viscosity of about 4,000 to 12,000 cps, one or more salts or buffers.

In an embodiment, the oxytocin is present in a powdered form. In the powdered formulation, oxytocin may be delivered as the only component, or other components may be mixed with the powder, such as magnesium or zinc salts, and the like. Dry powder forms of oxytocin are known in the art (See, for example, Fabio et al., AAPS PharmSciTech, vol. 16, no. 6, 1299-1306, 2015, incorporated herein by reference). For example, oxytocin powders preparations for inhalation may contain excipients and stabilizing agents, such as trehalose, isoleucine, polyvinyl-pyrrolidone, citrate (sodium citrate and citric acid), and zinc salts (such as zinc chloride and zinc citrate).

In an embodiment, a nasal spray formulation is made as follows: for a total quantity of 38.0 mls, 0.3080 gm of oxytocin powder, 2.3 gm of magnesium chloride, 0.1080 gm of citric acid anhydrous granular, 2.15 gm of sodium citrate powder, 3.8 mls of PCCA Mucolox™ liquid, and water (Formulation 1). This provides 45 IU units of oxytocin per 0.1 ml of the nasal spray.

In another embodiment, the formulation is made as follows: for a total quantity of 30.0 mls, 0.0.45 gm of oxytocin 10 units/mg trituration, 1.83 gm of magnesium chloride crystals, 0.0860 gm of citric acid anhydrous granular, 1.7 gm of sodium citrate powder, 3.0 mls of PCCA Mucolox™ liquid, and water (Formulation 2). This provides 15 units IU units of oxytocin per 0.1 ml of the nasal spray.

According to an embodiment, this disclosure provides a method for treating VSS. The method comprises administering intranasally to an individual a composition comprising or consisting essentially of oxytocin, wherein such administration results in treatment of VSS. The effectiveness of treatment of VSS may be monitored by quantifying VSS, such as by self-evaluation using clinically accepted techniques. An example is the Visual Snow Handicap Inventory (VSHI), a validated and published measurement technique. (van Dongen R M, Alderliefste G J, Onderwater GLJ, Ferrari M D, Terwindt G M. Migraine prevalence in visual snow with prior illicit drug use (hallucinogen persisting perception disorder) versus without. Eur J Neurol. 2021;28:2631-2638.). Adapted from the Tinnitus Handicap Inventory, the VSHI is a 25-point scale assessing perceived disability caused by VSS. In an embodiment, the improvement in an individual using VSHI may comprise an improvement of at least 10%. In various embodiments, the improvement may comprise from 10-100%, such as at least 20, 30, 40, 50, 60, 70, 80, 90, 95, or 99%.

In an embodiment, the method comprises administering about 50 to 260 IU of oxytocin over a 24 hour period. In an embodiment, the method comprises delivering about 100 to 250 IU, 160-240 IU, or 50 to 200 IU over a 24 hour period. In various embodiments, the oxytocin delivered may be 50, 75, 100, 125, 150, 175, 200, 225, and 250 IU over a 24 hour period. The oxytocin amount over a 24 hour period may be delivered in 2-5 doses. In an embodiment, the doses may be from 1 to 6 or 2 to 6 or more. In an embodiment, it is delivered in 3, 4, or 5 doses. Each dose may deliver the same or different amount of oxytocin as the other doses. In an embodiment, each dose may deliver from 12 to 65 IU per dose.

The results of the present disclosure were unexpected because using lower amounts of oxytocin (such as under 20 IU per day) did not result in any significant relief. The addition of a mucoadhesive polymer results in having to use fewer doses per day and reduces the flow-out of the sprayed composition.

The present compositions may be delivered to any part of the nasal cavity. In an embodiment, the compositions may be delivered to the vascular rich upper nasal cavity. For example, devices may be used that specifically deliver compositions to the upper nasal cavity. This enables active agents to rapidly enter the circulation, and particularly the central nervous system. Devices developed for delivery to the upper nasal cavity include those described in U.S. Pat. Nos. 9,550,036 and 9,919,117 to Impel Neuropharma Inc. (incorporated herein by reference). The compositions delivered using these devices may be liquid-based or may be in a dry powdered form. In an embodiment where the compositions are delivered to the upper nasal cavity, the mucoadhesive polymer may not be necessary. In an embodiment, a composition comprising or consisting essentially of oxytocin and optionally salts and/or buffer, and containing no thickener may be delivered to the upper nasal cavity. The amount of oxytocin delivered to the upper nasal cavity over a 24 hour period may be less than what is required if delivered as to the outer nasal cavity. For example, the oxytocin delivered to the upper nasal cavity may be from 50 to 260 IU or 50 to 200 IU of oxytocin over a 24 hour period or may be less than 50 IU, such as, for example, 20 to 200 or 20 to 50 IU over a 24 hour period. In embodiments, the oxytocin delivered may be 20 to 260 IU, 50 to 250 IU, 50 to 225 IU, 100 to 225, 20 to 225 IU, and all values and ranges between 20 and 260 IU over a 24 hour period.

Individuals that can be treated with the present compositions include those who are suffering from VSS. While the present method is useful for those individuals who are afflicted with VSS but do not suffer from migraine, the method can also be used for those individuals in whom VSS is accompanied by migraine. The present compositions are useful for treatment of VSS in male subjects and non-pregnant female subjects.

### EXAMPLES

The following are some non-restrictive examples of the embodiments of this disclosure.

Example 1. A method of treating VSS comprising administering intranasally to an individual in need of treatment from 50 to 260 IU of oxytocin over a 24 hour period.

Example 1a. The method of Example 1, wherein the individual is administered from 50 to 250, 50 to 225, 50 to 200, 100 to 225, 100 to 250, or 100 to 200 IU of oxytocin over a 24 hour period.

Example 1b. The method of Example 1a, wherein the individual is administered from 100 to 200 IU or 100 to 225 of oxytocin over the 24 hours period.

Example 1c. The method of Example 1b, wherein the individual is administered from 160 to 200 IU of oxytocin over the 24 hour period.

Example 1c.1 The method of Example 1c, wherein the individual is administered about 180 IU of oxytocin over the 24 hour period.

Example 1d. The method of any of the preceding Examples, wherein the oxytocin is delivered in 3, 4, or 5 doses over the 24 hour period.

Example 1e. The method of Example 1d, wherein each dose delivers from about 12 to 65 IU of oxytocin.

Example 1f. The method of Example 1, wherein oxytocin is delivered in a composition which further comprises a thickener present in an amount of from 5% to 12% by weight of the composition, said thickener having a viscosity of from 4,000 to 12,000 cps.

Example 1g. The method of Example 1, wherein the administration is continued for days, weeks, or months.

Example 1h. The method of Example 1, wherein the individual is a male or a post-menopausal female.

Example 1i. The method of Example 1, wherein the individual is also suffering from migraine.

Example 1j. The method of Example 1, wherein the individual is not suffering from migraine.

Example 2. A sprayable composition for intranasal administration comprising about 100 to 600 IU of oxytocin per ml, and a thickener present in an amount of from 5% to 12% by weight of the composition, said thickener having a viscosity of from about 4,000 to 12,000 cps.

Example 2a. The sprayable composition of Example 2, wherein the composition comprises about 150 to 450 IU of oxytocin per ml.

Example 2b. The sprayable composition of Example 2, wherein the thickener is Mucolox™.

Example 2c. The sprayable composition of Example 2 further comprising salts and/or buffers.

Example 3. A sprayable unit dose of oxytocin formulated for intranasal administration comprising from 15 to 65 IU of oxytocin and a thickener present in an amount of from about 5% to 12% by weight of the composition, said thickener having a viscosity of from about 4,000 to 12,000 cps.

Example 3a. The sprayable unit dose of Example 3, wherein the volume of the dose is about 0.05 to 0.15 ml, such as 0.1 ml.

Example 3b. The sprayable unit dose of Example 3, wherein the thickener is Mucolox™.

Example 4. A pharmaceutical composition suitable for intranasal administration comprising oxytocin, and a viscosity regulating agent present in an amount of from about 1% to about 15% by weight of the composition.

Example 4a. The pharmaceutical composition of Example 4, wherein the viscosity regulating agent is Mucolox™ present in an amount of from 5% to 12%.

Example 4b. The pharmaceutical composition of Example 4 or 4a, wherein oxytocin is present in an amount of 0.05 to 0.1% by weight.

Example 4c. The pharmaceutical composition of any of Example 4-4b, further comprising salts and/or buffers.

Example 4d. The pharmaceutical composition of any of Example 4-4b, further comprising magnesium chloride, citric acid, and sodium citrate.

Example 5. A pharmaceutical composition for intranasal administration comprising or consisting essentially of:
a) oxytocin present in an amount of 0.05 to 0.1% by weight;
b) a thickener in an amount of about 5% to 12% by weight, said thickener having a viscosity of about 4,000 to 12,000 cps, and
c) one or more salts or buffers.

Example 5a. The pharmaceutic composition of Example 5, wherein the salts or buffers are magnesium chloride, sodium citrate, and citric acid.

Example 6. A pharmaceutical composition for intranasal administration comprising or consisting essentially of:
a) from 100 to 600 IU of oxytocin per ml,
b) a thickener present in an amount of from 5% to 12% by weight of the composition, said thickener having a viscosity of from about 4,000 to 12,000 cps, and
c) magnesium chloride, citric acid, and sodium citrate.

Example 7. A method of treating VSS in an individual comprising delivering to the upper nasal cavity of the individual oxytocin in a powdered or aerosolized form.

Example 7a. The method of Example 7, wherein the oxytocin is delivered in a powdered form at an amount of about 20 to 200 IU of oxytocin over a 24 hour period.

Example 7b. The method of Example 7a, wherein the oxytocin powder is at least 95% pure oxytocin.

Example 8. A method of treating VSS in an individual comprising delivering to the upper nasal cavity of the individual oxytocin present in a sprayable composition, in one or more doses, from 20 to 260, 20 to 225, or 20 to 200 IU of oxytocin over a 24 hour period.

Example 8a. The method of Example 8, wherein delivery is achieved by propelling the composition into the upper nasal cavity.

Example 8b. The method of Example 8, wherein the oxytocin composition contains a mucoadhesive polymer.

Example 9. Compositions for use in any of the methods from any of the preceding examples.

The following example is provided to further illustrative the invention and is not intended to be restrictive in any way.

This example describes clinical use of the present formulations to treat VSS. In a patient suffering for VSS (who had failed to respond to treatment with lamotrigine, verapamil or acetazolamide), an initial dose of less than 20 IU daily was tried, but this did not provide any relief. A compounded formulation of oxytocin that provided a dose of 60 IU daily (30 IU twice daily) was then used. The formulation contained oxytocin, magnesium chloride, citric acid, and sodium citrate in sterile water. The patient reported partial benefit. The dose was then increased to 45 IU (3 sprays of 15 IU) 4 times daily. This produced a significant relief. A thickener was then added to the formulation keeping the oxytocin amount the same. The patient then reported several days of VSS-free period. Her score on the VSHI reduced from a pretreatment score of 40 to a current score of 20. The patient discontinued treatment because she was unable to use the spray 4 times daily. Upon cessation of therapy, the VSS returned to pre-treatment levels.

In another patient with VSS, an oxytocin dose of 45 IU twice daily was found to be of significant benefit. This patient has reported that his VSS symptoms reduced by 50% but never totally resolved. He remained improved on this dosing schedule for 3 months, but discontinued dosing because of the frequency required.

While the present invention has been described through illustrative embodiments, routine modification will be apparent to those skilled in the art and such modifications are intended to be within the scope of this disclosure.

What is claimed is:

1. A method of treating visual snow syndrome comprising administering intranasally to an individual in need of treatment from 50 to 260 IU of oxytocin over a 24 hour period.

2. The method of claim 1, wherein the oxytocin is delivered in a composition which further comprises a thickener present in an amount of from 5% to 12% by weight of the composition, said thickener having a viscosity of from 4,000 to 12,000 cps.

3. The method of claim 1, wherein the oxytocin is in a powdered or aerosolized form and is delivered to the upper nasal cavity of the individual.

4. The method of claim 1, wherein the oxytocin is delivered in a composition which further comprises a mucoadhesive polymer.

5. The method of claim 1, wherein the oxytocin is delivered in 3 to 5 doses over the 24 hour period.

6. The method of claim 5, wherein each dose delivers from 12 to 65 IU of oxytocin.

7. A method of treating visual snow syndrome in an individual, the method comprising delivering to the upper nasal cavity of the individual 20 to 225 IU of oxytocin over a 24 hour period.

8. The method of claim 7, wherein the oxytocin is delivered in a composition which further comprises a thickener present in an amount of from 5% to 12% by weight of the composition, said thickener having a viscosity of from 4,000 to 12,000 cps.

9. The method of claim 7, wherein the oxytocin is delivered in a powdered or aerosolized form.

10. The method of claim 7, wherein the oxytocin is delivered in a composition which further comprises a mucoadhesive polymer.

11. The method of claim 7, wherein the oxytocin is delivered in 3 to 5 doses over the 24 hour period.

12. A pharmaceutical composition for treating visual snow syndrome, the pharmaceutical composition comprising 100 to 600 IU of oxytocin per ml and a thickener present in an amount of from 5% to 12% by weight of the composition.

13. The pharmaceutical composition of claim 12, wherein the thickener comprises a viscosity of from 4,000 to 12,000 cps.

14. The pharmaceutical composition of claim 12, wherein the composition is in a powdered or aerosolized form and is delivered to the upper nasal cavity of an individual.

15. The pharmaceutical composition of claim 12, wherein the composition further comprises a mucoadhesive polymer.

16. The pharmaceutical composition of claim 12, wherein the composition further comprises magnesium chloride, citric acid or sodium citrate.

17. The pharmaceutical composition of claim 12, wherein the composition is delivered in 3 to 5 doses over a 24 hour period.

18. A sprayable unit dose for treating visual snow syndrome, the sprayable unit dose being formulated for intranasal administration and comprising 15 to 65 IU oxytocin.

19. The sprayable unit dose of claim 18, wherein the sprayable unit dose further comprises a thickener present in an amount of from about 5% to 12% by weight, said thickener having a viscosity of from about 4,000 to 12,000 cps.

20. The sprayable unit dose of claim 18. the sprayable unit dose comprising a volume of from 0.05 to 0.15 ml.


SEQUENCE LISTING (1 sequence)

SEQ ID NO: 1 - protein, 9 aa, Homo sapiens
CYIQNCPLG